(19) United States
(12) Patent Application Publication
(10) Pub. No.: US 2023/0125644 A1
(43) Pub. Date: Apr. 27, 2023

(54) DELIVERY DEVICE

(71) Applicant: TecMed AG, Burgdorf (CH)

(72) Inventors: Ursina Streit, Kirchberg (CH); Jan Baumert, Grünen (CH); Thomas Buri, Burgdorf (CH); Michael Hanimann, Bern (CH); Roland Margot, Worb (CH); Fabian Steiner, Burgdorf (CH); Patrick Hostettler, Hasle (CH); Simon Martin Bosshard, Hindelbank (CH); Martin Brügger, Bolligen (CH)

(21) Appl. No.: 17/972,692

(22) Filed: Oct. 25, 2022

(30) Foreign Application Priority Data
Oct. 25, 2021 (EP) 21204488.7

(51) Int. Cl. A61M 5/142

(52) U.S. Cl. CPC A61M 5/14248; A61M 5/14216; A61M 2005/14252

(57) ABSTRACT

A delivery device for administering a drug includes a housing, a reservoir and a drive device with a movable piston rod. The piston rod is guided through an aperture in an inner or outer wall of the housing. The aperture is sealed but allows the piston rod to move. The shape of the circumference of the cross-section of the piston rod is approximately a non-trivial curve of constant width.

### CROSS-REFERENCE TO RELATED APPLICATIONS

This application claims priority to European Patent Application No. 21204488.7 filed Oct. 25, 2021, entitled “IMPROVED DELIVERY DEVICE”, which is incorporated herein by reference in its entirety and for all purposes.

### TECHNICAL FIELD

The present disclosure relates to the field of delivery devices and delivery devices for delivering fluid drugs, such as infusion or injection devices, and provides delivery devices with movable piston rods.

### BACKGROUND

A variety of delivery devices for administering fluid, especially liquid drugs, are known. These range from simple disposable syringes to injection pens and infusion devices with sophisticated functions. In the case of reusable delivery devices, to maintain the longevity of these devices it is important to protect key device components such as the drive or electronics from exposure to moisture, e.g., from penetrating liquid.

The problem is explained herein, using insulin pumps as an example. However, the problem also occurs with reusable injection pens and other delivery devices, which is why the following explanations should not be interpreted as restrictive.

For example, the applicant's YpsoPump® is known from the prior art. The YpsoPump® is a conventional insulin pump with which insulin can be administered from a standard cartridge. FIGS. 1 to 3 show the entire YpsoPump® and portions thereof.

FIGS. 1 to 3 are taken from EP 3110475 B1, which is herein incorporated by reference in its entirety.

FIG. 1 shows an isometric view of the YpsoPump® p1. The housing p5 includes a viewing window p6 through which through which the status of the cartridge p2 (see FIG. 2) can be checked. Also shown are the control button p10 and the display p20, where the display p20 is a touch display. The infusion set adapter p30 and the infusion set tube p31 are also shown.

FIG. 2 shows a cross-section through the YpsoPump® p1 and the infusion set adapter p30. FIG. 2 is used to briefly explain how the YpsoPump® p1 operates. The cartridge p2 has an open end, which is closed by the movable plug p4. At its second end, the cartridge p2 is closed with a septum p3. The cannula p30a of the infusion set adapter p30 can pierce this septum, whereby medication (insulin) can pass through the cannula p30a into the infusion set tube p31 and ultimately be administered subcutaneously to the patient. The plug p4 is moved by the piston rod p52. FIG. 2 shows a full cartridge p2 and correspondingly the piston rod p52 fully retracted. The piston rod p52 is driven by the motor p40, which is connected to the piston rod p52 via the gear p45 and the drive sleeve p51. The piston rod p52 is connected to the drive housing p50 via the anti-rotation device p50a, which is non-rotatable but axially displaceable. Since the drive housing p50 is fixed in the housing p5, the piston rod p52 is also secured against rotation with respect to the housing p5. The drive sleeve p51 has a thread on its inner side, which is threadedly coupled to the proximal end of the piston rod p52. If the drive sleeve p51 is rotated on its axis, the piston rod p52 is displaced proximally or distally due to the aforementioned existing anti-rotation lock.

FIG. 2 further shows a battery compartment p91 with battery p90, negative terminal p93 and positive terminal p92. The positive terminal p92 is part of the battery compartment cover p7. FIG. 2 also symbolically shows the electronics p80.

The infusion set adapter p30 holds the cartridge p2 in the infusion pump p1 and is fixed in the YpsoPump® p1 via a bayonet catch. However, the infusion set adapter does not provide a watertight seal to the cartridge compartment p9.

In order to protect sensitive areas of the YpsoPump® p1, such as the electronics p80 or motor p40, from the ingress of liquids, various sealing elements, in particular O-rings p54 and p60, are arranged in the YpsoPump® p1, which protect the drive side/cartridge compartment. O-rings p92a and p93 also provide protection on the battery compartment side. The sliding engagement between piston rod p52 and anti-rotation device p50a is also not liquid-tight, for instance at the closed end p50b, so that the area between piston rod p52 and drive sleeve p51 could become contaminated—a potential for further improvement.

As shown in FIG. 3, there are longitudinal guide grooves p52a for the anti-rotation device of the piston rod p52, in which the anti-rotation device p50a (cam) engages. This groove/cam anti-rotation device makes it difficult to position an elastic sealing element in this area, which also functions as a bearing, which seals and at the same time also allows a sliding movement of the piston rod p52 within the pump p1.

Alternatively, piston rods with an angular cross-section (square, rectangle) are known to achieve an anti-rotation of the piston rod. But even with these classic shapes, a sealing problem arises because the contact pressure of a seal arranged around the cross-section reaches a maximum in the corners and is weaker along the edges, so that the risk of leaks is considerable.

### SUMMARY

Disclosed are delivery devices operated with conventional piston rods, which provide improved protection of device components from exposure to liquids.

Implementations relate to fluid drug delivery devices as defined herein for delivery of drugs or products. The delivery devices may be injection devices, for example injection pens, or infusion devices. In the case of infusion devices, these may include insulin pumps and patch pumps; and in the case of injection devices, these may include auto-injectors, pens for the automatic and repeated delivery of individually adjustable doses (so-called autopens) and patch injectors. The delivery devices may be of monolithic or modular configurations. Common to all devices, according to the present disclosure, is that there are areas of the delivery device that are to be protected from liquids. These may be mechanical, electrical, electronic, magnetic, or electromagnetic components, assemblies or combinations thereof.

A delivery device, according to the present disclosure, may include a housing, which in turn may, but need not, include several modules. The modules may be smaller housings in themselves.

In the housing of the delivery device, a reservoir may be located at least partially inside the housing. For example, a distal end of the reservoir through which the drug is delivered may be located outside the housing. The reservoir may include an internal volume, which is able to be reduced for dispensing the drug.

The reservoir may be a so-called cartridge in the broader sense, including a dispensing or shot end with a septum, the septum being pierceable by means of a cannula. For example, the cannula may be a hypodermic needle, or the cannula of an infusion set adapter. At its opposite end, the cartridge may be configured to be open, with a movable stopper or plug closing the open end. Thus, an internal volume is formed in the cartridge, and this volume may be increased or decreased by moving the stopper. Various cartridge materials are known to the skilled person, and may be constructed of glass or plastic, with a round or oval cross-section, or with a linear axis or a curved axis (e.g., toroidal).

Alternatively, the reservoir may be a bag that is squeezed when the drug is delivered.

The delivery device, according to the present disclosure, may further include a drive device. The drive device may be at least partially arranged in the housing or a module of the housing. The drive device may serve to expel the drug from the reservoir when the reservoir is present. The drive device may include a drive. The drive may serve as a source of mechanical energy. The drive may be a motor, such as an electric motor. Alternatively, and for instance when the delivery device is an injection device, the drive may be an arrangement of one or more springs. The function of the drive is to move a piston rod, which is also at least partially movably mounted in the housing or partially movably mounted in one or more modules. According to the present disclosure, the piston rod may be displaceably but non-rotatably mounted directly or indirectly in the housing or a module thereof. The drive may be directly or indirectly coupled to the piston rod. For instance, if the drive is a motor, a gear may be arranged between the drive and the piston rod, which may convert the motor movement (usually a rotating, driven axis) into a sliding movement of the piston rod. The coupling between the drive (direct or indirect) and the piston rod may, for example, occur via a threaded coupling between the drive and the piston rod. For example, the piston rod may have an internal thread in a threaded engagement with a threaded rod or spindle of the drive. Rotation of the threaded rod or spindle may cause or evoke displacement of the piston rod due to the non-rotating nature of the piston rod. The threaded rod or spindle may thus be the output element of a gear between a motor and the piston rod.

The piston rod may, for example, move a cartridge stopper or compress a bag during its displacement movement.

As described, the piston rod may be mounted movably, but rotation about its own axis relative to the housing or the module (e.g., housing module) in which the piston rod is mounted may not be permitted or even possible. According to the present disclosure, an aperture may be provided in an inner wall of the housing or a wall of a module of the housing, on or in which one or more anti-rotation elements are present. The piston rod may be guided through this aperture and mounted therein so that it is movable or slidably mounted. The aperture may be a polygonal aperture or passage through which the piston rod is guided, and the polygonal shape may approximately correspond to the cross-section of the piston rod. In this way, an anti-rotation feature may be provided in a manner similar to that described previously. In addition or alternatively, rotation of the piston rod may be prevented by providing additional elements or structures fixed to the aperture. The wall may also be mechanically reinforced in the area of the aperture to absorb additional forces acting on the wall. For instance increasing the wall thickness, providing rib-like reinforcements, or other means known to the skilled person may be used to provide anti-rotation features and/or structural reinforcement.

The aperture may further be provided with a seal according to the present disclosure, which may function to seal the area between the aperture and the piston rod, such that when the piston rod is passed through the aperture, no liquid can pass from one side of the wall to the other through the aperture. For instance, a bearing surface between the piston rod and the aperture may include the seal configured to prevent liquid from passing through the aperture, while permitting displacement of the piston rod. The seal may be formed with a material that is at least elastically deformable, and may function to seal the periphery of the piston rod at least via direct contact or by conforming to the piston rod, e.g., by forming a circumferential seal. For instance, the contact between the seal and the piston rod may not only be linear, but may also extend along the axis of the piston rod so that a circumferential seal is formed. The displaceability of the piston rod may be maintained and may for instance be movable bi-directionally or in multiple directions based on the operation of the delivery device.

According to the present disclosure, the piston rod may have a cross-sectional shape, at least over the axial area that is displaced through the aperture, adapted to facilitate providing an improved seal. The shape of the cross-section may correspond to a non-trivial “orbiform curve”. To illustrate what a curve of constant width is (also referred to as a curve of uniform thickness or equal thickness), the comprehensible definition from Wikipedia is reproduced here:

“In geometry, a curve of constant width is a simple closed curve in the plane whose width (the distance between parallel supporting lines) is the same in all directions. The shape bounded by a curve of constant width is a body of constant width or an orbiform . . . . These curves can also be constructed using circular arcs centered at crossings of an arrangement of lines, as the involutes of certain curves, or by intersecting circles centered on a partial curve.
Every body of constant width is a convex set, its boundary crossed at most twice: by any line, and if the line crosses perpendicularly it does so at both crossings, separated by the width. By Barbier's theorem, the body's perimeter is exactly π times its width, but its area depends on its shape, with the Reuleaux triangle having the smallest possible area for its width and the circle the largest. Every superset of a body of constant width includes pairs of points that are farther apart than the width, and every curve of constant width includes at least six points of extreme curvature. Although the Reuleaux triangle is not smooth, curves of constant width can always be approximated arbitrarily closely by smooth curves of the same constant width.
Cylinders with constant-width cross-section can be used as rollers to support a level surface. Another application of curves of constant width is for coinage shapes, where regular Reuleaux polygons are a common choice. The possibility that curves other than circles can have constant width makes it more complicated to check the roundness of an object.
Curves of constant width have been generalized in several ways to higher dimensions and to non-Euclidean geometry.”

The simplest, nota bene trivial curve of constant width is the circle. The simplest non-trivial curve of constant width, which represents a cross-sectional shape according to the present disclosure, is the so-called Reuleaux triangle or arc triangle r1, as shown in FIG. 4a. FIG. 4a further shows the equilateral triangle r2, which serves to construct Reuleaux triangle r1, as well as the radius R, which corresponds to the width of the curve of constant width and the side length of the triangle. FIG. 4d shows a triangular curve of constant width with rounded corners r5; FIGS. 4b and 4c show the construction of the same. The corners are rounded with the radius r4, where the size of the radius |r4| corresponds to the additional application to the entire curve of constant width, so that the resulting width of the curve of constant width can be calculated as R′=R+2*|r4|. r3 corresponds to the radius R plus |r4|, thus only one times |r4|. Here the size of the radius r3 can assume any size |r3|, where |r3|>R, resulting in |r4| from |r3|−R.

Between the Reuleaux triangle and the circle, there are an infinite number of other curves of constant width; what they have in common with the Reuleaux triangle is the odd number of corners and the convex shape between the corners. For instance, the curve of constant width may include three or more corners such as five corners or seven corners. As a further example, the pentagonal curve of constant width and its construction are shown in FIGS. 5a to 5d, where FIG. 5d shows the finished curve of constant width r10; FIGS. 5b and 5c show the construction of the same. FIG. 5a shows the pentagon r11, which serves as the starting point for the construction of the pentagonal curve of constant width. R2 is the radius for the circular arcs r12 and corresponds to the thickness of the uniform thickness or constant width r10.

Curves of constant width are not required to be equilateral like the Reuleaux triangle. The construction simply follows certain mathematical rules. The convex curved sides are central in the sealing of the aperture, whereby more regular pressure distributions in the seal (or surface pressure acting on the piston rod) can be accomplished. Nevertheless, such cross-sectional shapes make it possible to achieve anti-rotation.

In aspects of the present disclosure, the area of the wall that includes the aperture may be configured as a two-component injection-molded part, where the supporting component, e.g., the actual wall, may be injection molded from a first material (e.g., a first component, which may be referred to as a pre-molded part such as plastic, for instance a rigid thermoplastic polymer) and the seal component may be injection molded from a second material, which may be at least elastically deformable (e.g., a second component) for instance relative to the first material. On the one hand, this approach makes it possible to provide an area of the wall in one operation with a suitable seal. On the other hand, the area of the wall may contain further apertures, which may also be sealed in the same operation. These can for instance be seals for operating elements (buttons) or seals for electrical feed-throughs.

In some aspects, the material for the second component may be injection-moldable, such as a thermoplastic polymer, such as thermoplastic polyurethane or thermoplastic polyamide. The thermoplastic polymer may also be a thermoplastic elastomer. In a further alternative, the second component may include silicone, for example a two-part silicone, which cures in the injection molding device.

In aspects of the present disclosure, the delivery device may be an infusion pump in the style of the YpsoPump® as described herein. Alternatively, the infusion pump may be a modular pump including, for example, a reusable module with electronics and drive as well as a disposable module. The disposable module may then contain, for example, the drug reservoir and, for instance, an energy source. The infusion pump may be a so-called patch pump, which may be adhered to the skin of the person using it, and may be configured as a modular patch pump.

In aspects, the delivery device may be an injection device. For instance, the injection device may be pen- or pencil-shaped. Alternatively, the injection device may be a so-called patch injector, which may be adhered to the skin of the user for a single injection of a drug. In a further alternative, the injection device may be a pen-shaped auto-injector, such as that marketed by the applicant as Ypsomate™. In yet another alternative, the injection device may be an injection pen configured to automatically dispense multiple doses, such as the applicant's well-known ServoPen®. The injection devices may, for example, contain electronics that require protection.

### BRIEF DESCRIPTION OF THE DRAWINGS

Implementations are described in connection with the appended figures, which are exemplary and are in no way to be interpreted as limiting.

FIGS. 1 to 3 show the prior art according to the preceding explanations provided in the Background.

FIGS. 4 and 5 show details of the various forms of the curve of constant width, according to the present disclosure provided in the Summary.

FIGS. 6a to 10e show an embodiment according to the present disclosure.

FIG. 11 shows an alternative embodiment.

More specifically, in the drawings:

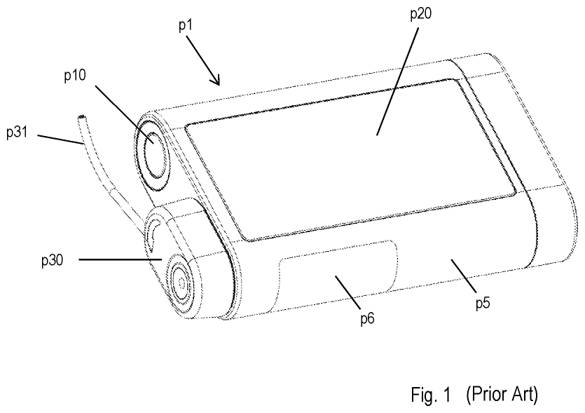

FIG. 1 Prior art: YpsoPump®;

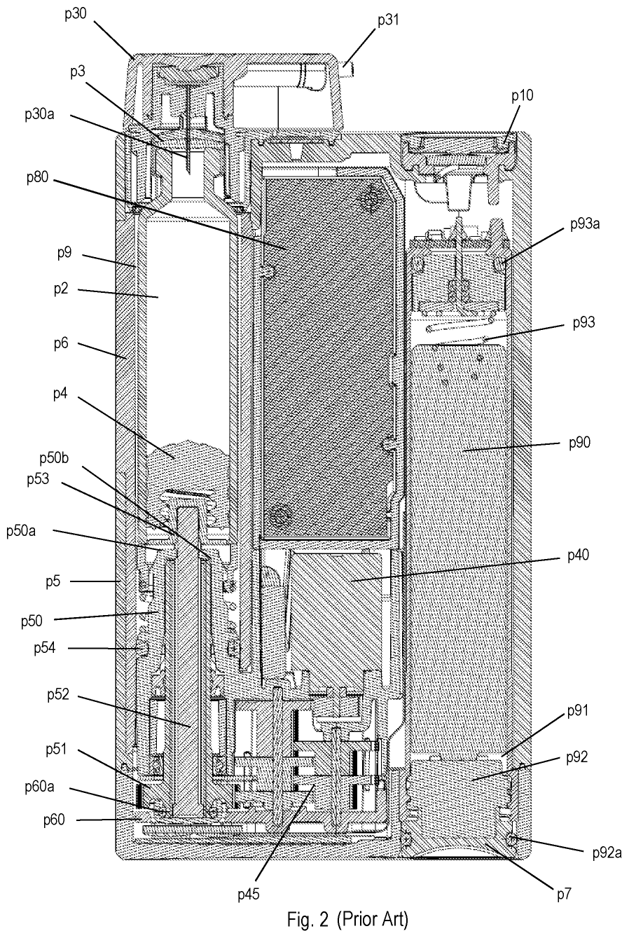

FIG. 2 Prior art: Longitudinal cross-section through YpsoPump® with seals;

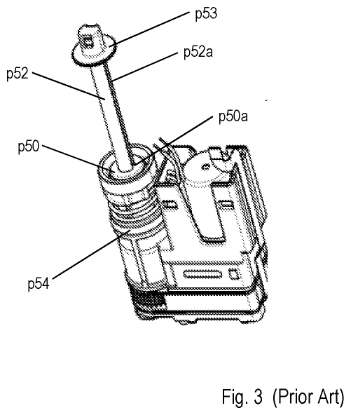

FIG. 3 Prior art: Drive unit with a piston rod and a seal;

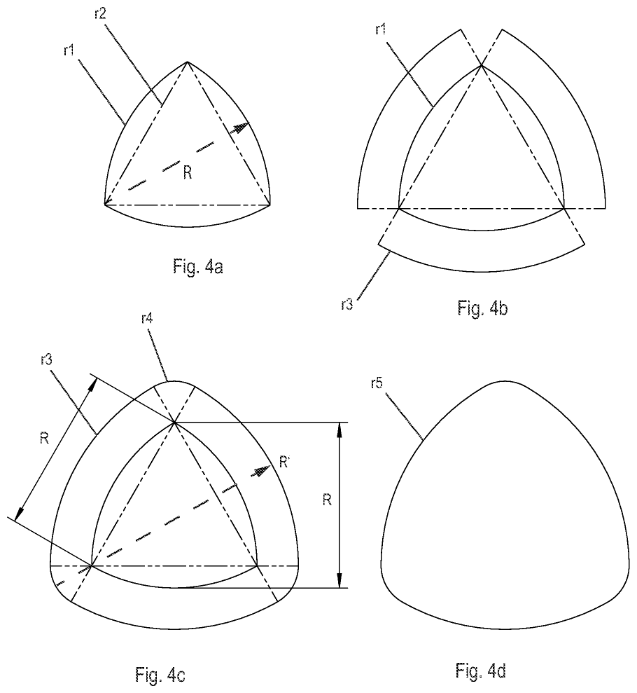

FIGS. 4a, 4b, 4c, and 4d Reuleaux triangles and rounded Reuleaux triangles;

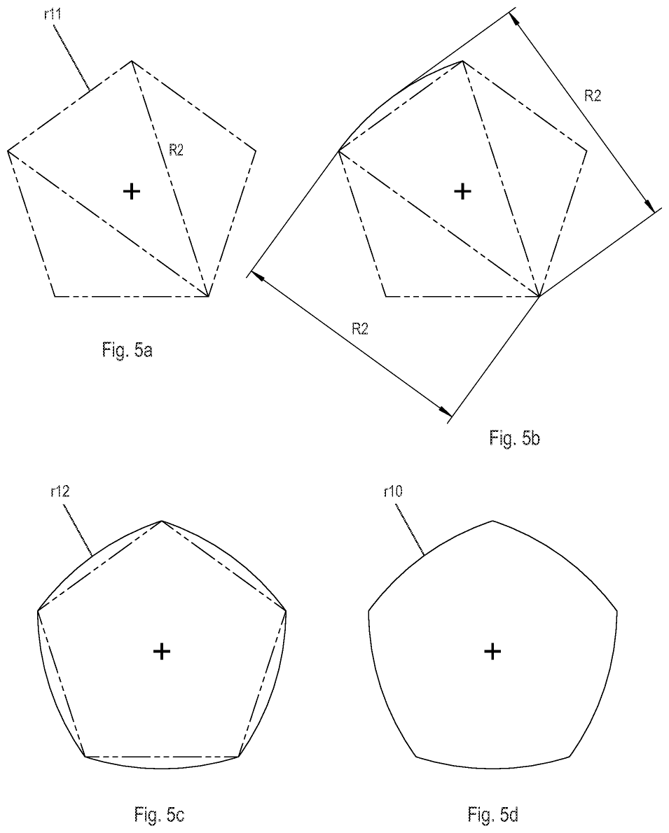

FIGS. 5a, 5b, 5c, and 5d Pentagonal curves of constant width and construction thereof;

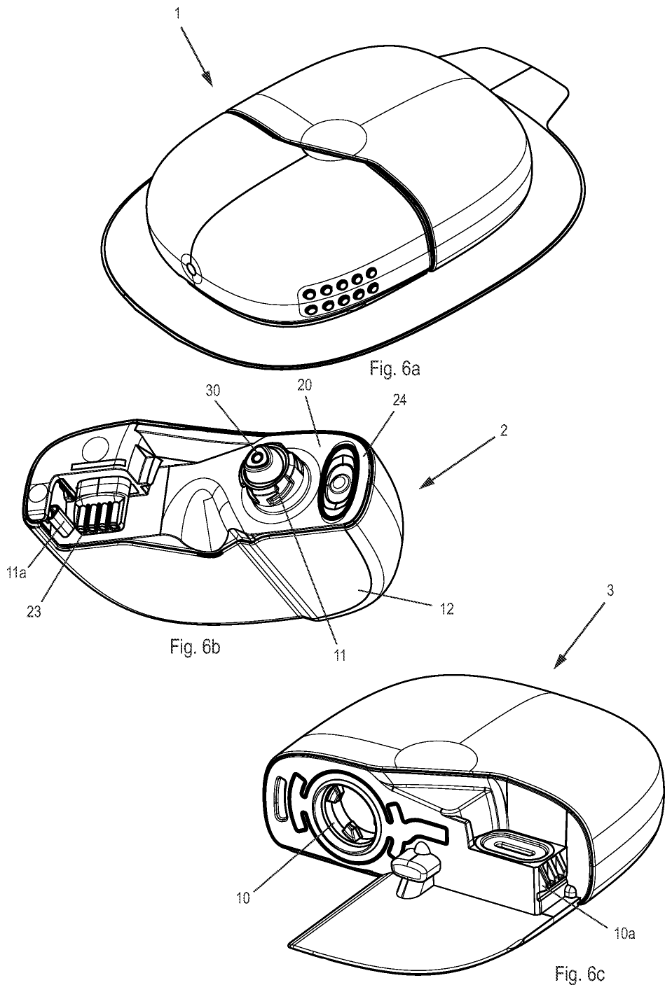

FIG. 6a Isometric view of an infusion pump according, to the present disclosure configured as a modular infusion pump;

FIG. 6b Pump module of the infusion pump of FIG. 6a, according to the present disclosure;

FIG. 6c Reservoir module of the infusion pump of FIG. 6a, according to the present disclosure, where the patch is not shown;

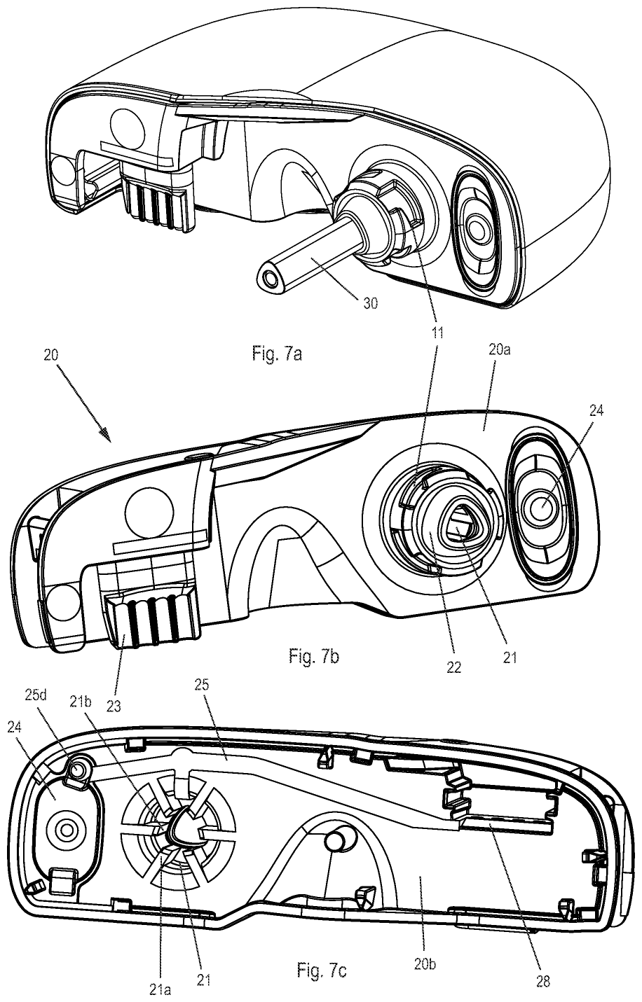

FIG. 7a Pump module of the infusion pump of FIG. 6a, according to the present disclosure, with an extended piston rod, where the cross-section of the piston rod corresponds to a curve of constant width with rounded corners;

FIG. 7b Front element of the pump module of FIG. 7a, showing an exterior of the front element;

FIG. 7c Front element of the pump module of FIG. 7a, showing an interior of the front element;

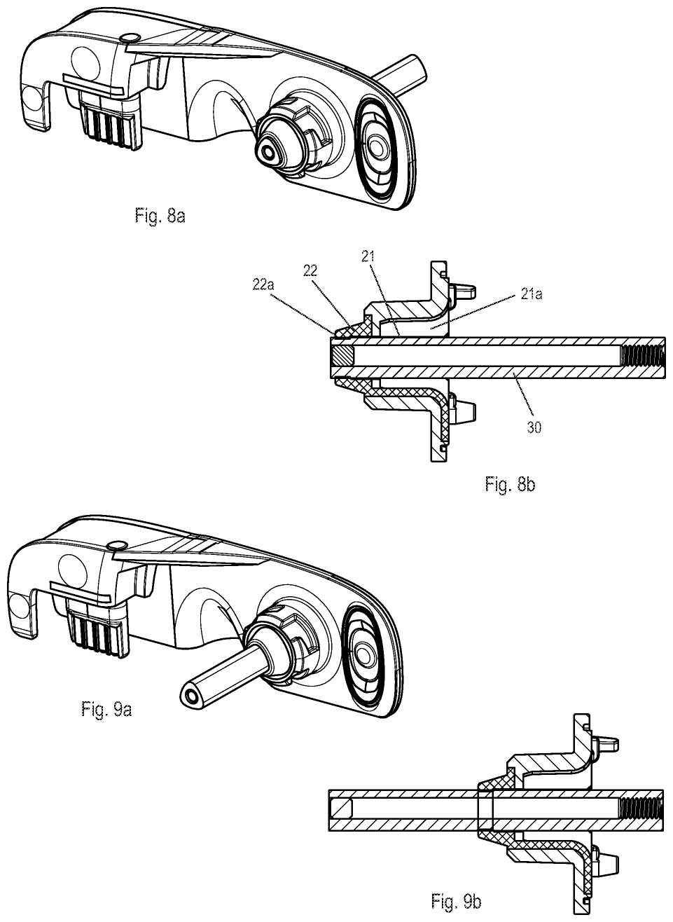

FIG. 8a Front element of the pump module of FIG. 7a showing an inserted piston rod in a retracted position;

FIG. 8b Cross-section through the front element and the inserted piston rod in the state of FIG. 8a;

FIG. 9a Front element of the pump module of FIG. 7a showing the inserted piston rod in an extended position;

FIG. 9b Cross-section through the front element and the inserted piston rod in the state of FIG. 9a;

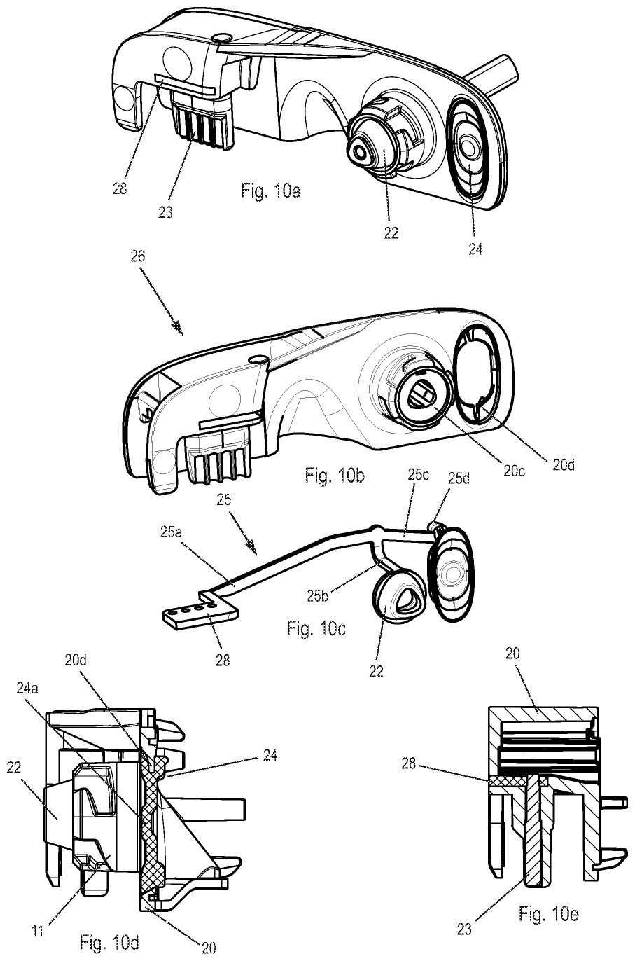

FIG. 10a Front element of the pump module of FIG. 7a with an inserted piston rod with a two-component injection-molded part;

FIG. 10b showing a hard, pre-molded component of the two-component injection molded part;

FIG. 10c showing a sealing component of the two-component injection molded part;

FIG. 10d Cross-section through the front element of FIG. 10a in the area of a reset button;

FIG. 10e Cross-section through front element of FIG. 10a in the area of the electrical plug connection; and

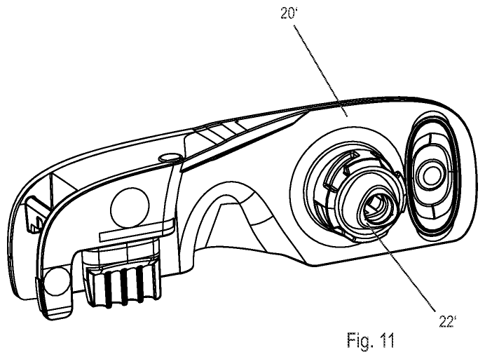

FIG. 11 Alternative embodiment of the front element.

### DETAILED DESCRIPTION

The term “product”, “drug” or “medicinal substance” in the present disclosure includes any flowable medicinal formulation suitable for controlled administration by means of a cannula or hollow needle into subcutaneous or intramuscular tissue, for example a liquid, solution, gel or fine suspension containing one or more medicinal active ingredients. A drug may thus be a composition containing a single active ingredient or a premixed or co-formulated composition containing multiple active ingredients from a single container. In particular, the term includes medicaments such as peptides (e.g. insulins, drugs containing insulin, preparations containing and derived from GLP 1 or analogous preparations), proteins and hormones, biologically derived or active ingredients, active ingredients based on hormones or genes, nutritional formulations, enzymes and other substances both in solid (suspended) or liquid form. The term also includes polysaccharides, vaccines, DNA or RNA or oligonucleotides, antibodies or parts of antibodies, and appropriate base, auxiliary and carrier substances.

The term “distal” means a side or direction towards the front, insertion end of the delivery device or towards the tip of the injection needle. In contrast, the term “proximal” means a side or direction towards the rear end of the delivery device opposite to the insertion end.

The terms “delivery device” and “delivery equipment” are used synonymously in this document.

FIG. 6a shows a delivery device according to the present disclosure in the form of a patch pump 1. The patch pump 1 is similarly constructed as described in the European patent application EP20181599.0, published as EP 3928811 A1, which is hereby incorporated by reference in its entirety.

Details of the basic technology of the patch pump 1 can be taken directly and unambiguously from the aforementioned published application.

Patch pump 1 may be of modular configuration, as shown in FIGS. 6b and 6c, and may include a drive module or pump module 2 and a reservoir module 3, which may be detachably connected to each other via a bayonet fitting including a bayonet catch 10 on the reservoir module 3, and a bayonet catch 11 on the pump module 2. When the reservoir module 3 and the pump module 2 are connected to each other via the bayonet fitting, the latching spring 11a of the pump module 2 engages behind the latching lug 10a of the reservoir module 3 so that the modules 2, 3 cannot detach from each other unintentionally. The pump module 2 may be reusable and may include electronics, a drive (e.g., with a motor, a gear, and transmission elements) as well as a piston rod 30 movably mounted therein. Due to its inclusion of the drive, the pump module 2 may also be referred to herein as a drive module. Furthermore, the pump module 2 may include an energy source such as a rechargeable battery.

The reservoir module 3 may include the reservoir, a power source in the form of, for example, a battery, and an infusion line that may be configured to deliver the drug to be administered from the reservoir to the tissue. When the reservoir module 3 and the pump module 2 are connected, the power source may be used to charge the energy source of the patch pump 2 (e.g., the rechargeable battery or an analogous energy storage device such as a capacitor). The reservoir may generally have the shape of a cartridge with a movable plug supported in the reservoir. By moving the plug in the reservoir, the volume in the reservoir may be increased or decreased. When the pump module 2 and reservoir module 3 are connected, the plug in the reservoir of the reservoir module 3 may be moved by an axial movement of the piston rod 30 of the drive module. For instance, a movement of the piston rod 30 into the reservoir module 3 may cause a reduction of the volume in the reservoir and finally a release of the drug through the infusion line into the tissue of the person using it.

The piston rod 30 may be movable or slidable, such as partially moved or slid out of the pump module 2. The pump module 2 may include a housing 12 and a face element or front element 20. Various components may be arranged on the front element 20, as shown for example in FIGS. 6b and 7a. These may include but are not limited to a bayonet catch 11 (e.g., a component of the pump module 2, which may be arranged around the aperture 21, which may be nozzle-shaped), an aperture 21 for the piston rod 30, a plug for the electrical connector 23 and a reset button 24. The front element 20 includes a front side 20a and a rear side 20b, see FIGS. 7b and 7c, respectively.

The piston rod 30 may be guided in the aperture 21, see, e.g., FIGS. 7a to 7c. According to the present disclosure, the piston rod 30 may include a cross-section, which may correspond to the shape of a triangular curve of constant width with rounded corners. The aperture 21 may include a shape, e.g., a complementary shape, which corresponds approximately to the negative of the curve of constant width of the piston rod 30, so that the piston rod 30 can be moved through the aperture 21, but cannot be rotated around the axis of the piston rod 30. The piston rod 30 may therefore be slidably mounted in the aperture 21 so that it can move but is secured against rotation. The aperture 21 may be reinforced by ribs 21a, where the ribs 21a with their end faces 21b may also guide the piston rod 30 (see, e.g., FIGS. 8a to 9b). In order to protect the interior of the pump module 2 from liquids, a seal 22 may be arranged on the front side 20a of the front element 20. A sealing surface 22a of the seal 22 may follow the shape of the curve of constant width of the cross-section of the piston rod 30 (see e.g., FIGS. 8b and 9b) and form a circumferential seal. In the undeformed state, the aperture of the seal 22 may be smaller than the cross-section of the piston rod 30. If the piston rod 30 is pushed through the aperture of the seal 22, the aperture is expanded. Therefore, the seal 22 may be constructed of a deformable material, such as an elastomer. The seal 22 may thus prevent liquid from penetrating the interface between the piston rod 30 and the seal 22 and thus the aperture 21. The seal 22 may be bonded to the bayonet catch 11 of the bayonet fitting portion, for instance at or to the front face 20c of the front element 20. According to the present disclosure, the housing 12 may be constructed of plastic, and the front element 20 may be configured as a two-component injection molded part (e.g., a two-shot injection molded part), which may allow the wall of the front element 20 and the seal 22 to be manufactured as one part with a tight bond, e.g., an adhesive bond. As shown in FIGS. 10b and 10c, the front element 20 may include a component 26, referred to as a face plate 26, and a seal component 25. To facilitate understanding of the front element 20, the front element 20 has been divided into its individual components in FIGS. 10b and 10c. The seal component 25 may be constructed of a softer material (e.g., an elastomeric material) relative to the face plate 26 responsible for guiding the piston rod 30, among other things. For this purpose, the face plate 26 may require a certain rigidity and strength (e.g., a plastic material). During production, the face plate 26 may be injection molded first and then the seal component 25 may be injection molded in the same mold.

As shown in FIGS. 10c and 10d, the seal component 25 may include not only the seal 22, but also a seal 24 for the reset button. For instance, a reset switch may also be provided in the seal component 25, which may be provided to reset settings in the pump electronics of the pump module 2, for instance, to delete the corresponding memory and/or to restart the electronics of the pump module 2 (e.g., by a short power interruption). FIG. 10d shows a vertical cross-section through the front element 20 in the area of the reset button including the seal 24. The seal 24 may be a structured membrane, for instance where the operating element 24a of the seal 24 does not protrude from the surrounding wall of the front element 20 in the direction of the front 20a, but rather may lie flat or recessed to prevent unintentional actuation of the reset button. In order to hold the seal 24 firmly in the front element 20 (or to improve the adhesion), retaining elements 20d (see FIGS. 10b and 10d) may be arranged on the front element 20 at the transition between the seal 24 and the front element 20 (for instance, a chemical adhesive bond may exist). When the seal 24 is cast onto the face plate 26, this may establish an interlock between the face element 20 and the seal 24.

Furthermore, the seal component may additionally include a seal 28 for the electrical contacts of the electrical connector 23. In the embodiment shown, the seal 28 may be configured to be undersized compared to the electrical contacts of the electrical connector 23, for instance configured as pins, which may establish a radial contact pressure to seal against such pins or other electrical contacts. This may prevent liquid from penetrating into the interior of the pump module 2 along the electrical contacts. FIG. 10e shows a vertical cross-section through the face element 20 in the region of the electrical connector 23, the right of the figure being the front of the face element 20.

In implementations, the disclosed seals may be produced separately or together, and when produced together, the seals may be connected to each other via arms 25a, 25b and 25c. Corresponding channels may be provided on a back side of the face plate 26 for producing the adjoined seals. In such implementations, all seals may be molded on via a lug 25d. In this way, the end plate 20 with seals may be elegantly produced in one injection molding process such as a two-shot injection molding process.

FIG. 11 shows an alternative configuration of the front element 20 of the present disclosure in which a modified front element 20′ may include a modified seal 22′. In the modified seal 22′, the aperture may be configured to be circular and may not be based on the curve of constant width of the piston rod 30. Functional sealing by the seal 22 may nevertheless be possible, precisely due to the shape of the curve of constant width permitting a more homogeneous stress distribution in the seal 22′ so that, for example, in the area of the corners relative to the edges, no excessive stress difference arises, as it may otherwise arise with for example an equilateral triangle as a cross-section for the piston rod. Nevertheless, the shape of the curve of constant width, as described, may provide a non-rotatable piston rod secured against rotation due to the configuration of the delivery devices as provided herein.

### REFERENCE LIST

### Prior Art:

p1 YpsoPump®
p2 Cartridges
p3 Septum
p4 Plugs
p5 Housing
p6 Viewing window
p7 Battery cover
p9 Cartridge compartment
p10 (Control) button
p20 Touch display
p30 Infusion set adapter
p30a Cannula
p31 Infusion tube
p40 Motor
p45 Gear
p50 Drive housing
p50a Anti-rotation device
p51 Drive sleeve
p52 Piston rod
p52a Guide groove
p53 Flange
p54 Seal (O-ring)
p60 Bearing plate
p60a Seal
p90 Battery
p91 Battery compartment
p92 Positive battery terminal/connection
p92a Seal
p93 Negative battery terminal/connection
p93a Seal

### Curve of Constant Width Figures:

r1 Reuleaux triangle (simplest non-trivial curve of constant width)
R Width
r2 Equilateral triangle, as a basis for the construction of the Reuleaux triangle
r3 Extended arch for construction curve of constant width with rounded corners
r4 Corner radius (|r4| corresponds to the size of the radius)
R′ Width (R′=R+2*|r4|)
r5 Curve of constant width with rounded corners
r10 Curve of constant width with five corners
r11 Construction pentagon
r12 Circular arc of the pentagonal curve of constant width
R2 Width

### Delivery Devices:

1 Delivery device in the form of a modular patch pump
2 (reusable) pump module or drive module
3 Reservoir module
10 Bayonet catch (part of reservoir module 3)
10a Latching lug
11 Bayonet catch (part of reservoir module 2)
11a Latching spring
12 Pump module housing
20 Front element of the pump module 2, configured as a two-component injection molded part
20′ Alternative face element
20a Front side of face element 20
20b Rear side of face element 20
20c Front face
20d Retaining elements
21 Aperture
21a Ribs
21b Front faces
22 Piston rod seal
22′ Alternative piston rod seal
22a Sealing surface
23 Electrical connector element with electrical contacts
24 Seal reset button (actual reset button not shown)
24a Operating element
25 Seal ensemble (sealing component of the face element 20)
25a Sealing connection to seal 28 for the electrical contacts
25b Sealing connection to seal 22 for piston rod 30
25c Sealing connection to seal 24 for the reset button
25d Lug
26 Face plate (load-bearing component of the face element) 20
28 Seal for electrical contacts 23
30 Piston rod with the cross-sectional shape of a curve of constant width

What is claimed is:

1. A delivery device for delivering a fluid drug, comprising:
a housing comprising a reservoir module and a drive module configured to be detachably connected;
a reservoir configured with a variable internal volume for storing the fluid drug, wherein the reservoir is at least partially arranged in the reservoir module of the housing;
a drive device at least partially arranged in the drive module of the housing, the drive module comprising:
a drive, the drive comprising a motor or a spring; and
a movable piston rod mounted in the housing and configured to be movable by the drive to change the variable internal volume of reservoir,
wherein the piston rod is configured to be guided via an aperture through at least one of an inner wall of the housing, an outer wall of the reservoir module, or an outer wall of the drive module,
wherein the piston rod is displaceably and non-rotationally mounted in the aperture,
wherein a bearing surface between the piston rod and the aperture comprises an elastically deformable seal configured to prevent liquid from passing through the aperture while permitting displacement of the piston rod, and
wherein a shape of a circumference of a cross-section of the piston rod is approximately a non-trivial curve of constant width.

2. The delivery device according to claim 1, wherein the curve of constant width is a curve of constant width with three or more corners.

3. The delivery device according to claim 2, wherein the curve of constant width comprises five corners or seven corners.

4. The delivery device according to claim 1, wherein the curve of constant width is a curve of constant width with rounded corners.

5. The delivery device according to claim 1, wherein a housing of the reservoir module and a housing of the drive module are configured to be detachably connectable to each other, wherein at least a portion of the housing of the drive module is constructed of a two-component injection molded material in an area of the outer wall comprising the aperture, wherein a material of a first component defining the outer wall differs from a material of a second component defining the seal.

6. The delivery device according to claim 5, wherein the material of the second component is a thermoplastic elastomer.

7. The delivery device according to claim 6, wherein the thermoplastic elastomer comprises at least one of a thermoplastic polyurethane or a thermoplastic polyamide.

8. The delivery device according to claim 5, wherein the material of the second component is a silicone.

9. The delivery device according to claim 5, wherein a region of the outer wall comprising the aperture comprises at least one additional aperture comprising at least one additional seal, and wherein the at least one additional seal is constructed of the material of the second component of the two-component injection molded material.

10. The delivery device according to claim 9, wherein a seal component defines the seal and the at least one additional seal, wherein the seal and the at least one additional seal are coupled by at least one arm of the seal component.

11. The delivery device according to claim 9, wherein the at least one additional aperture is configured as an aperture for at least one of an operating element or for one or more electrical contacts.

12. The delivery device according to claim 1, wherein the delivery device is an infusion pump.

13. The delivery device according to claim 12, wherein the infusion pump is a patch pump.

14. The delivery device according to claim 1, wherein the delivery device is an injection device.

15. The delivery device according to claim 14, wherein the injection device is a pen-shaped injection device.
